(19) United States
(12) Patent Application Publication
(10) Pub. No.: US 2003/0220396 A1
(43) Pub. Date: Nov. 27, 2003

(54) Method for treating ocular hypertension and glaucoma

(75) Inventor: Ryuji Ueno, Montgomery, MD (US)

Correspondence Address: SUGHRUE MION, PLLC, 2100 PENNSYLVANIA AVENUE, N.W., WASHINGTON, DC, 20037, US

(73) Assignee: SUCAMPO AG

(21) Appl. No.: 10/397,243

(22) Filed: Mar. 27, 2003

Related U.S. Application Data
(60) Provisional application No. 60/367,722, filed on Mar. 28, 2002.

(51) Int. Cl. A61K 31/557

(52) U.S. Cl. 514/530; 514/573; 514/690

(57) ABSTRACT

The present invention provides a method for treating ocular hypertension and glaucoma, which comprises administrating an ophthalmic solution comprising as an active ingredient thereof 15-keto-prostaglandin compound having a ring structure at the end of the ω chain, wherein the intraocular pressure (IOP) lowering effect is improved by adjusting the osmolarity ratio of said solution to be within a specific range.

### TECHNICAL FIELD

The present invention relates to a method for treating ocular hypertension and glaucoma. In more detail, the invention relates to an ophthalmic solution for topical application to the eye, comprising as an active ingredient thereof 15-keto-prostaglandin compound having a ring structure at the end of the ω chain, wherein the osmolarity ratio of said solution is adjusted within a specific range.

### BACKGROUND ART

It is known that human lacrimal fluid has almost the same osmolarity as that of physiological saline or a 0.9 w/v % sodium chloride solution. In view of safety, Yakushin (Notification of Pharmaceuticals and Cosmetics division, Japan Pharmaceutical Affairs Bureau) 2, No. 667 prescribes that artificial tear type ophthalmic solution, which is required to have physical and chemical traits close to physiological conditions of eyes, should have the osmolarity ratio (the ratio to the osmolarity of physiological saline) between 0.85-1.55.

On the other hand, in an ophthalmic solution containing a chroman derivative, that is effective for treatment of diseases including diabetic keratopathy, the lower osmolarity ratio of the solution provides the better intraocular transition of the active ingredient. Accordingly, it is proposed to adjust the osmolarity ratio of the ophthalmic solution to 0.1-0.9, preferably to 0.3-0.6, more preferably to 0.4-0.5 (Japanese Patent Application Laid Open No. 130675/1999).

For conventional systemic treatment of glaucoma and ocular hypertension, a hyperosmotic composition that comprises mannitol (formulation for intravenous injection), concentrated glycerin or isosorbide (formulation for oral administration) has been used. Systemic administration of the hyperosmotic agent increases blood serum osmolarity, the increased blood serum osmolarity inhibits transition of water from the blood into the aqueous humor which causes inhibition of aqueous humor production and results in lowering the intraocular pressure (hereinafter, “IOP”) of the patient.

These hyperosmotic compositions are mainly used for treating an acute ocular hypertension attack due to an abrupt increase of the IOP after an ophthalmic operation.

However, with respect to an ophthalmic solution for treatment of glaucoma and ocular hypertension, it is not known how the osmolarity ratio of the solution affects the IOP lowering effect.

Prostaglandins (hereinafter, referred to as PG(s)) are members of class of organic carboxylic acids, which are contained in tissues or organs of human or other mammals, and exhibit a wide range of physiological activity. PGs found in nature (primary PGs) generally have a prostanoic acid skeleton as shown in the formula (A):

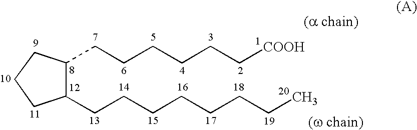

On the other hand, some of synthetic analogues of primary PGs have modified skeletons. The primary PGs are classified to PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGE₁ and PGE₂ and PGE₃ are known to have vasodilation, hypotension, gastric secretion decreasing, intestinal tract movement enhancement, uterine contraction, diuretic, bronchodilation and anti ulcer activities. PGF_{1α}, PGF_{2α}, and PGF_{3α}have been known to have hypertension, vasoconstriction, intestinal tract movement enhancement, uterine contraction, lutein body atrophy and bronchoconstriction activities.

Some 15-keto (i.e., having oxo at the 15-position instead of hydroxy)-PGs and 13,14-dihydro (i.e., having single bond between the 13 and 14-position)-15-keto-PGs are known as the substances naturally produced by the action of enzymes during the metabolism of primary PGs. It is also known that some 15-keto-PG compounds have IOP lowering effects and are effective for treatment of ocular hypertension and glaucoma (U.S. Pat. Nos. 5,001,153, 5,151,444, 5,166,178 and 5,212,200, all of which are incorporated herein by reference).

### DISCLOSURE OF THE INVENTION

The present inventor conducted an intensive study on the biological activity of 15-keto-prostaglandin compounds and found that IOP lowering effect of an ophthalmic solution for topical eye administration comprising as an active ingredient thereof 15-keto-prostaglandin compound having a ring structure at the end of the ω chain could be improved by adjusting its osmolarity ratio within a specific range, and has resulted in the completion of the present invention.

Namely, the present invention relates to a method for treating ocular hypertension and glaucoma, which comprises administrating an ophthalmic solution comprising as an active ingredient thereof a 15-keto-prostaglandin compound having a ring structure at the end of the ω chain to a subject in need of said treatment, wherein the osmolarity ratio of said solution is 0.5 or more.

The present invention further relates to an ophthalmic solution for treating ocular hypertension and glaucoma, which comprises as an active ingredient thereof a 15-keto-prostaglandin compound having a ring structure at the end of the ω chain, wherein the osmolarity ratio of said solution is 0.5 or more.

Furthermore, the present invention relates to use of a 15-keto-prostaglandin compound having a ring structure at the end of the ω chain for manufacturing an ophthalmic solution for treating ocular hypertension and glaucoma, wherein the osmolarity ratio of said solution is 0.5 or more.

In the present invention, the “15-keto-prostaglandin compound” (hereinafter, referred to as “15-keto-PG compound”) may include any of derivatives or analogs (including substituted derivatives) of a compound having an oxo group at 15-position of the prostanoic acid skeleton instead of the hydroxy group, irrespective of the configuration of the five-membered ring, the number of double bonds, presence or absence of a substituent, or any other modification in the a or ω chain.

The nomenclature of the 15-keto-PG compounds used herein is based on the numbering system of the prostanoic acid represented in the above formula (A).

A preferred compound used in the present invention is represented by the formula (I):

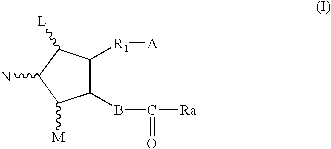

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, —CH₂OH, —COCH₂OH, —COOH or a functional derivative thereof;

B is —CH₂—CH₂—, —CH═CH— or —C≡C—;

R₁ is a saturated or unsaturated bivalent lower or medium aliphatic hydrocarbon, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon, which is substituted at the end by cyclo(lower)alkyl, cyclo(lower)alkyloxy, aryl, aryloxy, heterocyclic group or hetrocyclic-oxy group; wherein the aliphatic hydrocarbon is optionally substituted by halogen, oxo, hydroxy, lower alkyl, lower alkoxy or lower alkanoyloxy.

A group of particularly preferable compounds among the above-described compounds is represented by the formula (II):

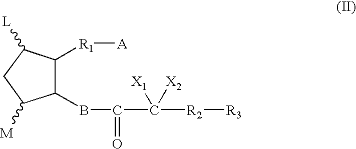

wherein L and M are hydrogen, hydroxy, halogen, lower alkyl, hydroxy(lower)alkyl, lower alkanoyloxy or oxo (wherein at least one of L and M is a group other than hydrogen, and the five-membered ring may have at least one double bond);

A is —CH₃, —CH₂OH, —COCH₂OH, —COOH or a functional derivative thereof;

B is —CH₂—CH₂—, —CH═CH—, —C≡C—;

X₁ and X₂ are hydrogen, lower alkyl, or halogen;

R₁ is a saturated or unsaturated bivalent lower or medium aliphatic hydrocarbon, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂ is a single bond or lower alkylene; and

R₃ is cyclo(lower)alkyl, cyclo(lower)alkyloxy, aryl, aryloxy, heterocyclic group or heterocyclic-oxy group.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 6 to 10 carbon atoms for R₁ and 1 to 10, especially 1 to 8 carbon atoms for R_{α.}

The term “halogen” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-i-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, and xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen atom, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydroxy which provides a 5-membered ring structure of, so called, PGF type.

Preferred A is —COOH, its pharmaceutically acceptable salt, ester or amide thereof.

Preferred B is —CH₂—CH₂—, which provide the structure of, so-called, 13,14-dihydro type.

Preferred examples of X₁ and X₂ include hydrogen and halogen, and preferably, both of them are hydrogen or at least one of them is halogen. A compound wherein both of X₁ and X₂ are halogen, especially fluorine that provides a structure of, so called 16,16-difluoro type is also preferable.

Preferred R₁ is a hydrocarbon containing 1-10 carbon atoms, preferably, 6-10 carbon atoms. Further, at least one of carbon atom in the aliphatic hydrocarbon is optionally substituted by oxygen, nitrogen or sulfur.

Examples of R₁ include, but not limited to, the following groups:

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—CH(CH₃)—CH₂—,

—CH₂—CH₂—CH₂—CH₂—O—CH₂—,

—CH₂—CH═CH—CH₂—O—CH₂—,

—CH₂—C≡C—CH₂—O—CH₂,

—CH₂—CH₂—CH₂—CH₂—CH₂—CH₂—CH₂—,

—CH₂—CH═CH—CH₂—CH₂—CH₂—CH₂,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH2—,

—CH₂—CH₂—CH₂—CH₂—CH₂—CH₂—CH═CH—,

—CH₂—C≡C—CH₂—CH₂—CH₂—CH₂—CH₂—,

—CH₂—CH₂—CH₂—CH₂—CH₂—CH₂—CH(CH₃)—CH_{2—}

Preferred Ra is a hydrocarbon containing 1-10 carbon atoms, more preferably, 1-8 carbon atoms which is substituted by aryl or aryloxy at the end.

The configuration of the five-membered ring and the α- and/or ω chains in the above formulae (I) and (II) may be the same as or different from that of the primary PGs. However, the present invention also includes a mixture of a compound having a primary type configuration and a compound of a non-primary type configuration.

The typical example of the present compound is 13,14-dihydro-15-keto-17-phenyl-18,19,20-trinor-prostaglandin F compound and its derivative or analogue.

The 15-keto-PG compound of the present invention may be in the keto-hemiacetal equilibrium by formation of a hemiacetal between hydroxy at position 11 and oxo at position 15.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the 15-keto-PG compounds used in the invention include both isomers. Further, while the compounds used in the invention may be represented by a structure formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324 and 5,739,161 and U.S. patent application Ser. No. 09011218 (these cited references are herein incorporated by reference).

The term “treatment” or “treating” used herein includes any means of control such as prevention, care, relief of the condition, attenuation of the condition, arrest of progression, etc.

The term “ophthalmic solution” as used herein represents a liquid type composition for topical application to the eyes, which may cover solution, emulsion, suspension and gel.

The “osmolarity ratio” as used herein represents a ratio of osmolarity of a sample solution to the osmolarity of physiological saline (i.e. NaCl 0.900 g in water 100 mL). Since the osmolarity of physiological saline is known and stable (286 mOsm), the osmolarity ratio of a sample solution may be calculated from the osmolarity (CT (mOsm) of the sample solution by the following equation

Osmolarity Ratio=C_{T}/C_{S}

wherein, Cₛ represents the osmolarity of 0.9% aqueous sodium chloride solution which is equal to 286 mOsm and C_{T} represents the osmolarity of the sample solution (mOsm).

The osmolarity of a sample solution is determined according to a conventional manner, for example, described in Japanese Pharmacopeia.

The ophthalmic solution may be manufactured according to any of conventional methods, for example, by dissolving the active ingredients in a sterile aqueous solution such as saline, buffering solution, or by combining powder compositions to be dissolved before use.

According to the present invention, the osmolarity of the ophthalmic solution is adjusted to 0.5 or more (osmolarity: 143 mOsm or more), preferably to approximately 0.5-1.5 (osmolarity: 143-429 mosm), more preferably to approximately 0.7-1.3 (osmolarity: 200-372 mOsm), further more preferably, to approximately 0.8-1.3 (osmolarity: 229-372 mOsm), and with special preference given to a solution of which osmolarity is adjusted to approximately 1 (osmolarity: 286 mOsm).

In order to adjust the osmolarity of the solution, any of conventional osmolarity modifiers used in the field of ophthalmology may be used as far as it is not contrary to the objects of the present invention. Examples of osmolarity modifiers may include, but not limited thereto, sodium chloride, potassium chloride, calcium chloride, sodium bicarbonate, sodium carbonate, magnesium sulfate, sodium hydrogen phosphate, sodium dihydrogen phosphate, dipotassium hydrogen phosphate, boric acid, borax, sodium hydroxide, hydrochloric acid, mannitol, isosorbitol, propylene glycol, glucose and glycerin,

The ophthalmic solution of the invention may further comprise an additive which is ordinary used in the ophthalmic field as desired. Examples of the additives may include buffering agent such as boric acid, sodium monohydrogen phosphate and sodium dihydrogen phosphate, preservatives such as benzalkonium chloride, benzethonium chloride and chlorobutanol, thickeners such as saccharide including lactose, mannitol or maltose, hyaluronic acid or its salt such as sodium hyaluronate and potassium hyaluronate, mucopolysaccharide such as chondroitin sulfate, sodium polyacrylate, carboxyvinyl polymer and crosslinked polyacrylate.

The present ophthalmic solution may be formulated as a sterile unit dose type product containing no preservatives.

The ophthalmic solution of the present invention may contain a single active ingredient or a combination of two or more active ingredients. In a combination of plural active ingredients, their respective dose may be suitably increased or decreased in consideration of their therapeutic effects and safety.

The concentration of the active ingredients in the ophthalmic solution and the frequency of administration may vary according to the compound to be used, the type of subject, age, weight, and symptom to be treated, desirable therapeutic effect, administration volume, period for treatment and the like. Although an optimal concentration may be chosen as desired, a typical ophthalmic solution containing 0.0001-10 w/v% of the active ingredient may be provided and used according to the invention. The typical frequency of instillation may be at least once daily.

Further, the ophthalmic solution of the invention may suitably contain another pharmacologically active ingredients, as far as they are not contrary to the object of the present invention.

The present invention will be described in more detail with reference to the following examples, which, however, is not intended to limit in any means the scope of the present invention.

### EXAMPLES

In the following examples, the osmolarity was measured by means of OSMOMETER (Model OM-801, VOGEL) at room temperature.

### Example 1

Two kinds of test ophthalmic solutions each containing 0.001 w/v% of test substance 1 (13,14-dihydro-15-keto-17-phenyl-18,19,20-trinor-PGF_{2α}isopropyl ester), which is 15-keto-prostaglandin compound having a ring structure at the end of the ω chain were prepared. The osmolarity ratio of one solution was adjusted to 0.7 (osmolarity: 200 mOsm) and the other was adjusted to 1.0 (osmolarity: 286 mOsm). Nine cynomolgus monkeys were used for the example. One of the test solutions was instilled (30 μL/eye) into one eye of the monkey, and one week after, the other test solution was instilled (30 μL/eye) into the same eye. The intraocular pressure (IOP) of the animals were measured with an applanation tonometer immediately before and 2, 4, 8, 12 and 24 hours after the instillation of the test solution. ΔIOP•AUC₀₋₂₄ₕ (area under the curve) was calculated based on the IOPs at each measurement time represented as change of IOPs(ΔIOPs) from that measured just before the instillation(time 0) The greater ΔIOP•AUC₀₋₂₄ₕ represents the greater IOP lowering effect.

Table 1 shows the result. The ophthalmic solution containing test substance 1 with the osmolarity ratio 1.0 exhibited significantly greater IOP lowering effect than the ophthalmic solution containing the same test substance 1 with the osmolarity ratio 0.7.

TABLE 1

| | Osmolarity | | [symbol] IOP · AUC_{0-24 h} |
|---|---|---|---|
| Test Substance | Ratio | N | Mean ± S.E. |
| Test Substance 1: 0.001% | 0.7 | 9 | 22.8 ± 11.8 |
| Test Substance 1: 0.001% | 1.0 | 9 | 55.1 ± 10.7* |

Test substance 1: 13,14-dihydro-15-keto-17-phenyl-18,19,20-trinor-PGF_{2α} isopropyl ester
*p < 0.05 Compared with 0.001% solution of test substance 1 with the osmolarity ratio 0.7 (paired Student's t-test)

### Example 2

Three kinds of ophthalmic solutions each containing 0.001 w/v% of test substance 1 were prepared and used. The osmolarity ratio of the solutions were adjusted to 0.8 (osmolarity: 229 mOsm), 1.0 (osmolarity: 286 mOsm) and 1.3 (osmolarity: 372 mOsm) respectively. Eight cynomolgus monkeys were used for this example. Test solutions were instilled to the monkey in the same manner as Example 1, i.e. 30 μL/eye of each solution was serially instilled to same eye with one-week intervals between the instillations. The IOP of the animals were measured with an applanation tonometer immediately before and 2, 4, 8, 12 and 24 hours after the instillation of each test solution. ΔIOP•AUC₀₋₂₄ₕ (area under the curve) was calculated based on the IOPs at each measurement time represented as change of IOPs(ΔIOPs) from that measured just before the instillation(time 0) The greater ΔIOP•AUC₀₋₂₄ₕ represents the greater IOP lowering effect.

Table 2 shows the result. The ophthalmic solution containing test substance 1 with the osmolarity ratio 1.0 exhibited greater IOP lowering effect than those with the osmolarity ratio 0.8 and 1.3.

TABLE 2

| | Osmolarity | | [symbol] IOP · AUC_{0-24 h} |
|---|---|---|---|
| Test Substance | Ratio | N | Mean ± S.E. |
| Test Substance 1: 0.001% | 0.8 | 8 | 41.1 ± 18.7 |
| Test Substance 1: 0.001% | 1.0 | 8 | 62.3 ± 19.3 |
| Test Substance 1: 0.001% | 1.3 | 8 | 37.9 ± 14.8 |

### COMPARATIVE EXAMPLE

Three kinds of ophthalmic solutions each containing 0.05 w/v% of test substance 2 (13,14-dihydro-15-keto-20-ethyl-PGF_{2α}isopropyl ester), which is a 15-keto-prostaglandin compound having a linear structure at the end of the ω chain, were prepared and used. The osmolarity ratio of the solutions were adjusted to 0.3 (osmolarity: 86 mOsm), 0.7 (osmolarity: 200 mOsm) and 1.0 (osmolarity: 286 mOsm), respectively. Each of the test solutions (35 μL/eye) was instilled serially into one eye of a white rabbit with one-week intervals between the installations. The IOP of the animals were measured with an applanation tonometer immediately before and 1, 2, 3, 4, 5 and 6 hours after the instillation. ΔIOP•AUC₀₋₆ₕ (area under the curve) was calculated based on the IOPs at each measurement time represented as change of IOPs(ΔIOPs) from the value of just before the instillation(time 0) The greater ΔIOP•AUC₀₋₆ₕ represents the greater IOP lowering effect.

Table 3 shows the result. Each ophthalmic solutions containing test substance 2 with the osmolarity ratio of 0.3, 0.7 and 1.0 exhibited substantially the same IOP lowering effect.

TABLE 3

| | Osmolarity | | [symbol] IOP · AUC_{0-6 h} |
|---|---|---|---|
| Test Substance | Ratio | N | Mean ±+01 S.E. |
| Test Substance 2: 0.05% | 0.3 | 6 | 18.9 ± 4.4 |
| Test Substance 2: 0.05% | 0.7 | 6 | 22.5 ± 5.1 |
| Test Substance 2: 0.05% | 1.0 | 6 | 20.3 ± 4.6 |

Test substance 2: 13,14-dihydro-15-keto-20-ethyl-PGF_{2α} isopropyl ester

These results indicate that, while 15-keto-prostaglandin compound having a linear structure at the end of the ω chain exhibited no difference in the IOP lowering effect by the difference of osmolarity ratio, 15-keto-prostaglandin compound having a ring structure at the end of the ω chain exhibited a significant difference in the IOP lowering effect by the difference of osmolarity ratio of the eye drops containing said compound.

1. A method for treating ocular hypertension and glaucoma, which comprises administrating an ophthalmic solution comprising as an active ingredient thereof 15-keto-prostaglandin compound having a ring structure at the end of the ω chain to a subject in need of said treatment, wherein the osmolarity ratio of said solution is 0.5 or more.

2. The method as described in claim 1, wherein said 15-keto-prostaglandin compound is a compound represented by the following general formula (I): 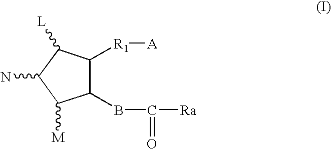
wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;
A is —CH₃, —CH₂OH, —COCH₂OH, —COOH or a functional derivative thereof;
B is —CH₂—CH₂—, —CH═CH— or —C≡C—;
R₁ is a saturated or unsaturated bivalent lower or medium aliphatic hydrocarbon, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon, which is substituted at the end by cyclo(lower)alkyl, cyclo(lower)alkyloxy, aryl, aryloxy, heterocyclic group or hetrocyclic-oxy group, wherein the aliphatic hydrocarbon is optionally substituted by halogen, oxo, hydroxy, lower alkyl, lower alkoxy or lower alkanoyloxy.

3. The method as described in claim 1, wherein said 15-keto-prostaglandin compound is a 13,14-dihydro-15-keto-prostaglandin compound.

4. The method as described in claim 1, wherein said 15-keto-prostaglandin compound is a 13,14-dihydro-15-keto-17-phenyl-18,19,20-trinor-prostaglandin compound.

5. The method as described in claim 1, wherein said 15-keto-prostaglandin compound is a 13,14-dihydro-15-keto-17-phenyl-18,19,20-trinor-PGF_{2α}isopropyl ester.

6. The method as described in claim 1, wherein the osmolarity ratio of the ophthalmic solution is about 0.5-1.5.

7. The method as described in claim 1, wherein the osmolarity ratio of the ophthalmic solution is about 0.7-1.3.
